(19) United States
(12) Patent Application Publication
(10) Pub. No.: US 2021/0162148 A1
(43) Pub. Date: Jun. 3, 2021

(54) AIRWAY INHALANT NEBULIZER CARTRIDGE

(71) Applicant: Nathan Christopher Maier, Hayward, CA (US)

(72) Inventor: Nathan Christopher Maier, Hayward, CA (US)

(21) Appl. No.: 16/726,087

(22) Filed: Dec. 23, 2019

Related U.S. Application Data
(63) Continuation-in-part of application No. 16/700,833, filed on Dec. 2, 2019.

(51) Int. Cl. A61M 15/00; A61M 11/02

(52) U.S. Cl. CPC A61M 15/0065; A61M 11/005; A61M 11/02

(57) ABSTRACT

A cartridge is inserted onto a nebulizer that aerosolizes (or vaporizes) liquid drawn from a cartridge reservoir via a flow path into a nebulization chamber. In such a nebulizer, an inlet port is coupled to an external air supply and leads through a Venturi nozzle into the chamber to direct an air stream across an opening of the flow path, while a discharge port leads from the chamber to a user mask, mouthpiece or cannula, where the aerosol or vapor mixture can be inhaled. A filtered outlet port isolates exhaled material from the external environment. Multiple discrete heating elements may be placed around the flow path to preheat the liquid. If the liquid is sufficiently volatile, heating may vaporize the material which can condense back into an aerosol after mixing with the air stream. A set of check valves direct one-way flow and prevent leakage or spillage of material from the cartridge or nebulizer. The cartridge and nebulizer can receive coded information for third-party activation by an authorized establishment.

### CROSS-REFERENCE TO RELATED APPLICATION

This is a continuation-in-part of prior U.S. patent application Ser. No. 16/700,833, filed Dec. 2, 2019.

### TECHNICAL FIELD

The present invention relates to cartridges for use with nebulizer/vaporizer devices for delivering nebulized aerosol or vapor to a patient for inhalation, e.g. for the administration of medication.

### BACKGROUND ART

In U.S. Pat. No. 5,603,314, Bono describes an aerosol inhalation device for delivering aerosol mist to a patient. The device comprises a nebulizer that generates and delivers an aerosol through a first conduit to the patient, and a filter that captures exhaled droplets received through a second conduit from the patient before passing now contaminant-free gas to an exhaust port.

In U.S. Patent Application Publication 2005/0263150, Chathampally et al. describes a system for administration of medications to a patient via a nebulizer in combination with an airtight face mask. The nebulizer, which is either an ultrasonic nebulizer or a jet nebulizer, produces a mist of medication-containing droplets. The nebulizer is connected to the face mask at a first one-way valve. A filtration unit, connected to the face mask at a second one-way valve, scavenges medications that would otherwise escape into the patient's immediate surroundings.

A problem with vaporizers is that its contents can be used by anyone, even when there are age or prescription requirements for their legitimate use. Counterfeit cartridges for the vaporizers filled with unknown and potentially dangerous substances may be sold to unwitting users. Even legitimate cartridges may be refilled with material other than their original contents and passed off as new. Still further, even where a cartridge contains the correct material, some users may overdose by overusing the nebulizer for too long or too often. Methods of controlling the use of vaporizers and their cartridges are needed.

### SUMMARY DISCLOSURE

A cartridge with a liquid reservoir connects to a nebulizer or vaporizer device. The liquid reservoir has a fluid outlet port with a first one-way valve that connects to a fluid flow path of the nebulizer or vaporizer device that will allow drawing of fluid contents from the reservoir, ultimately to be inhaled as aerosol or vapor by a user. The cartridge also has an air intake with a second one-way valve to admit air into the reservoir while preventing leakage or spilling of fluid from the reservoir.

A cartridge must first be activated by an authorized third-party establishment (a doctor's office, pharmacy, etc.) before its intended user can use it with the nebulizer. The cartridge is characterized by having a readable element that stores coded information, which is written at the time the cartridge is activated on behalf of the intended user by that authorized third-party establishment. The readable element may comprise a barcode or pin code on an edge of the cartridge. It might also comprise a writable RFID tag. Still further, it may comprise a flash memory powered by a direct pin interface through male/female headers, or by contactor spring and contact, such as microUSB or microSD with protocols defining data, data clock, and power channels of the bus configuration. The coded information could provide specifics about the cartridge's liquid contents needed by the nebulizer or vaporizer device, a manufacturer-specific authorization code, a cartridge batch identification code, a usage history log for the cartridge, a third-party establishment activation code, a biometric user identifier, or other information that can help prevent use of an unauthorized or refilled cartridge, a cartridge with nonapproved contents, or by an unauthorized user. A control circuit of the nebulizer can read such coded information to enable (or disable) the drawing of liquid contents from the reservoir into the nebulizer's flow path.

There are basically three main types of embodiment, a pure nebulizer, a hybrid nebulizer, and a, pure vaporizer, with which such a cartridge might be used. In a pure nebulizer, a source of highly pressurized air (from a pump or compressed air source) passes over a small Venturi nozzle at the opening or tip of the fluid flow path to nebulize liquid material drawn up through the flow path from a reservoir or cartridge. In a hybrid nebulizer, heating elements are provided around the fluid flow path to apply enough heat to lower the vapor pressure in the flow path so that effective nebulization is possible with liquids that would not otherwise be possible to nebulize, or to reduce the needed velocity of the air stream passing over the opening to cause the nebulization. In a pure vaporizer, enough heat could be added to vaporize the liquid material in the flow path so that nebulization by a high velocity air stream is not even necessary. In that case, the air stream simply serves to mix with the vapor and direct the mixture out of the chamber to the user.

### BRIEF DESCRIPTION OF THE DRAWINGS

FIG. 1 is side perspective view of a manually operated nebulizer device in accord with the present invention.

FIGS. 2 and 3 are two different perspective views from above of a second embodiment of a nebulizer in accord with the present invention.

FIG. 4 is a side elevational view of the second embodiment of FIGS. 2 and 3.

FIG. 5 is a partial open perspective view of the nebulization chamber in the embodiment of FIGS. 2 through 4.

FIG. 6 is a perspective view from above of a third embodiment of a nebulizer (or vaporizer) in accord with present invention that includes heating elements.

FIG. 7 is a perspective view of a liquid reservoir cartridge for use with any of the embodiments of the present invention.

FIGS. 8 and 9 are respective top plan and side perspective views of the third embodiment of the present invention.

FIG. 10A is a perspective view of liquid reservoir cartridge with a readable barcode or punch code. That code could provide information to a heater control circuit, such as optimal heating parameters (temperature, etc.) for the multiple discrete heating elements of the third embodiment, as well as manufacturer lot number, and authorization codes to prevent use of unapproved cartridges or reuse of refilled cartridges.

FIG. 10B is a perspective view of a liquid reservoir cartridge with a readable and writable RFID tag to provide the heater control circuit with the same kinds of information as the cartridge in FIG. 10.

FIGS. 11 and 12 are side sectional views of heated fluid flow paths for the third embodiment of the present invention, the first version in FIG. 11 using solid heating rings and the second version in FIG. 12 using heating coils.

FIGS. 13 and 14 are two different perspective cutaway views of the heated fluid flow paths for the third embodiment, which include a ball valve therein.

FIGS. 15 and 16A are respective side sectional views of two versions of a check valve and Venturi nozzle combination on an inlet port to any of the embodiments of the present invention.

FIG. 16B is sectional view of the nozzle taken along the line 16B-16B in FIG. 16A.

FIG. 17 is a block schematic diagram of a cartridge activation system.

FIG. 18 (divided into FIGS. 18A and 18B) is a flow diagram illustrating steps for third-party activation of a cartridge.

FIG. 19 (divided into FIGS. 19A and 19B) is a flow diagram illustrating steps for third-party activation of a nebulizer device.

FIG. 20 (divided into FIGS. 20A-20F) is a flow diagram illustrating steps for operation of an activated nebulizer device and cartridge.

### DETAILED DESCRIPTION

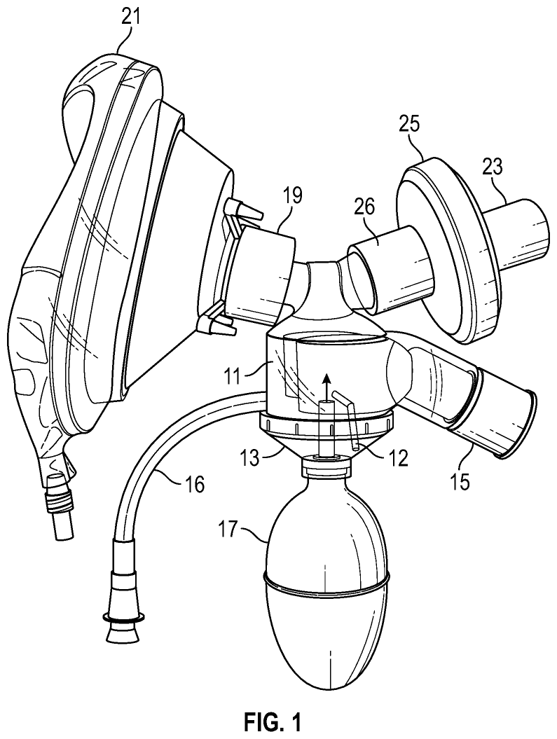

With reference to FIG. 1, a first embodiment of a nebulizer device, as described in co-pending prior U.S. patent application Ser. No. 16/700,833, comprises a nebulization chamber 11 with a liquid reservoir 13 connected via a fluid flow path 12 to the nebulization chamber 11, an inlet port and check valve 15 leading into the nebulization chamber 11 to provide a flow of accelerated air, in this case by means of a manually operated squeeze bulb 17, a discharge port 19 for aerosolized liquid leading through an air pathway into a user mask 21, and a filtered outlet port 23 from the user mask 21 for exhaled air, where the filter is contained within the enlarged volume 25. A check valve 26 in the outlet flow path prevents air being drawn in from the discharge side during inhalation. Internal features of the nebulization chamber 11 and of the various connecting pathways and ports are essentially as described below in more detail for the other embodiments, in that various one-way check valves are provided for the ports or pathways to minimize or eliminate any leakage of active liquid material and to ensure that the inhaled and exhaled air flow through the proper pathways, and in that accelerated air is directed across the opening of the fluid flow path 12 leading from the liquid reservoir to cause nebulization into an aerosol that can be inhaled by an patient through the mask 21. The mask 21 is sealed to ensure that inhaled material does not escape into the external environment. A mouthpiece or nasal cannula could also be used instead of the mask 21.

Instead of a squeeze bulb 17 to move air through the nebulization chamber 11, a hand or foot operated bellows could be provided, or a small gas canister, or (as in other embodiments described below) a pressurized air supply line. All these sources of accelerated air flow are functionally equivalent, and except perhaps for different sizes and proportions of internal features of the nebulization chamber 11 to ensure adequate flow velocity and efficient nebulization are substantially identical.

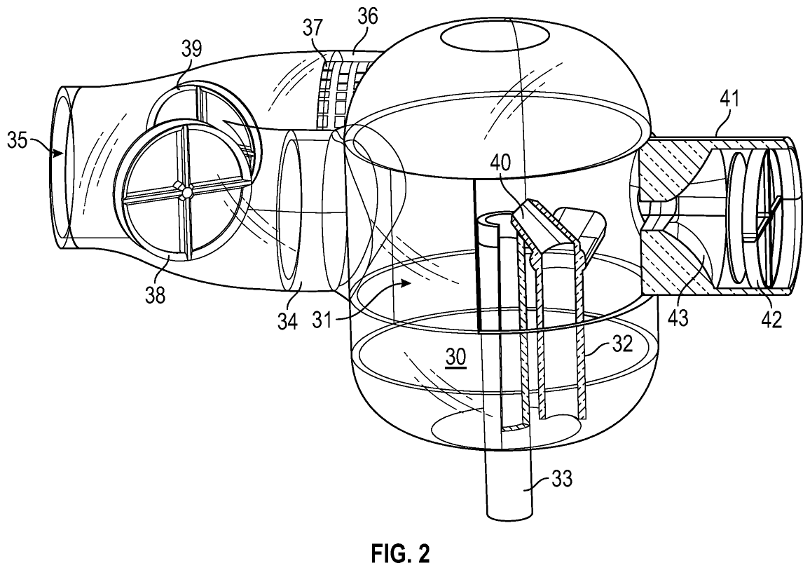

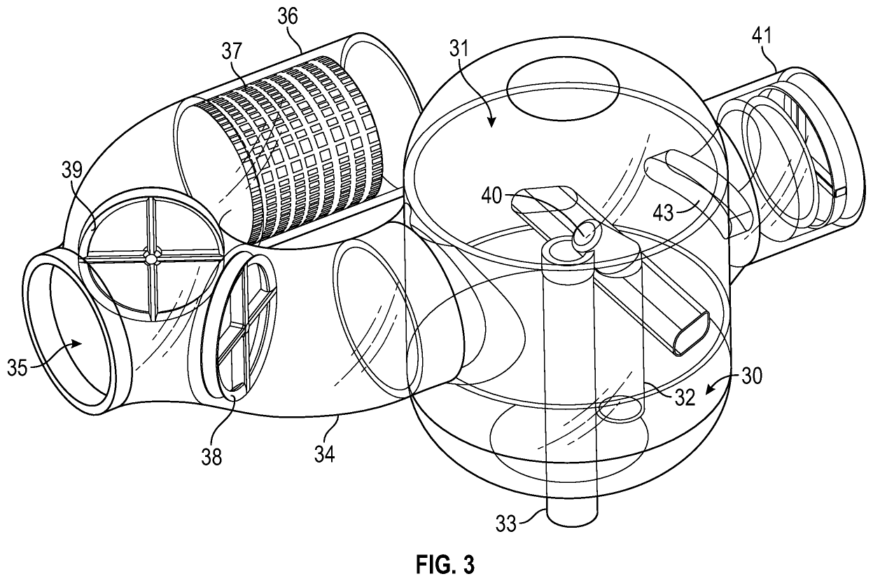

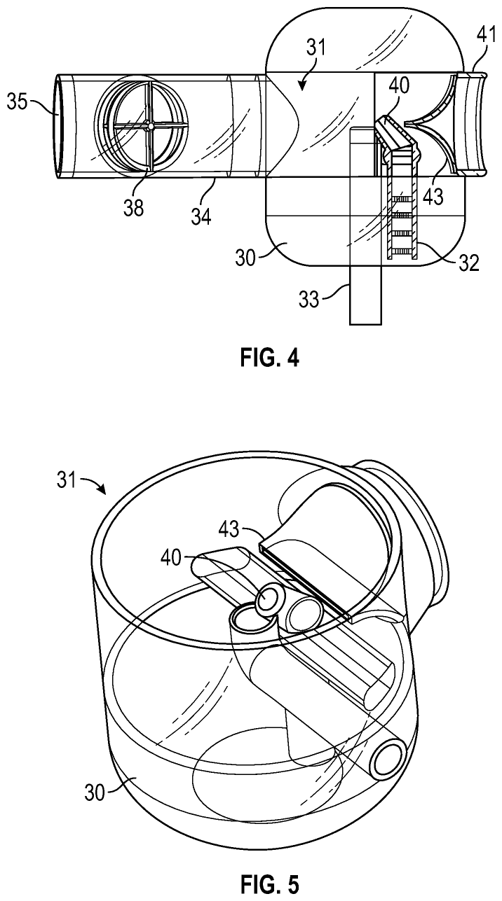

With reference to FIGS. 2-5, another embodiment of a nebulizer in accord with the invention illustrates in more detail a version of the internal components of a nebulization chamber 31. As in FIG. 1, there is a discharge port 34 leading from the chamber 31 through an air pathway 35 to a user mask, mouthpiece, or nasal cannula (not shown). A check valve 38 is provided for one-way flow of aerosol material from the discharge port 34 toward that mask. Likewise, there is an outlet port 36 containing a filter 37 (such as a HEPA filter or an activated charcoal filter), again with a check valve 39 providing one-way flow of exhaled air from the user mask to the outlet port 36.

In this embodiment, the bottom of the nebulization chamber 31 forms a liquid reservoir 30. A fluid flow path 32 extends from near the bottom of the reservoir 30 upwards to an opening 40. An inlet port 41 coupled to an external air supply leads through a check valve 42 and a Venturi nozzle 43 that directs a stream of accelerated air across the opening 40 of the fluid flow path 32. The nebulization device works with a non-pressurized air supply at atmospheric pressure, but a pressurized air supply could also be used, e.g. to assist those patients that have a compromised respiratory system. The check valve 42 serves mainly to prevent liquid in the chamber from leaking out in the event the nebulizer is tipped over.

Pressurized air source 33 provides high velocity air over the fluid path opening 40 to form very small droplets or mist. A stream of air enters through the inlet port 41 and is accelerated to high velocity by the Venturi nozzle 43. The high velocity air stream from the nozzle 43 carries the aerosolized material out of the chamber 31 through the discharge port 34 and to the user mask.

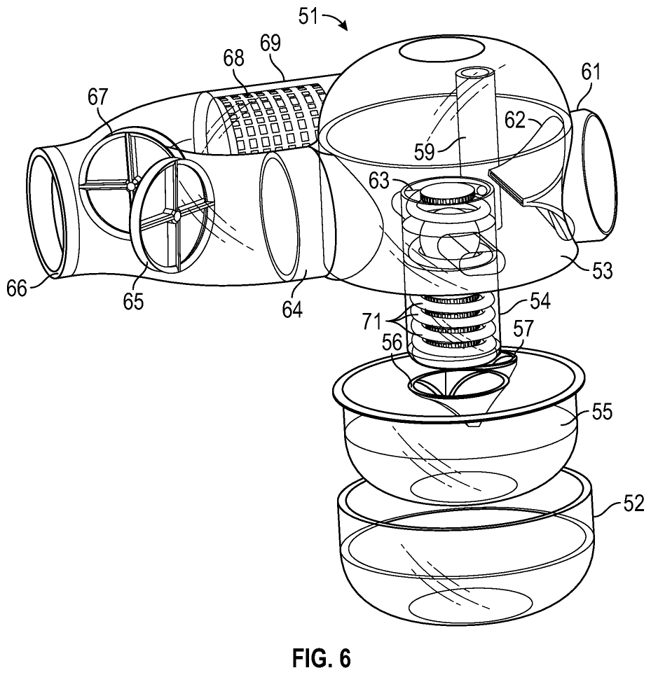

With reference to FIG. 6, another embodiment of a nebulizer in accord with the present invention, which may be either a hybrid (heat-assisted) nebulizer or a pure vaporizer device 51 (depending upon the liquid material and the amount of heating), features a heating system 71 around the fluid flow path 54 that applies heat to material drawn from the liquid reservoir 55 and flowing within the flow path 54. In the case of a hybrid nebulizer, the heating system 71 applies heat to the liquid in the flow path 54 to lower the vapor pressure so that nebulization can be effectively achieved with liquid materials that would not otherwise be possible with pure nebulizers as in FIGS. 1-5: In the case of a pure vaporizer, as opposed to a pure nebulizer or a hybrid (heat-assisted) nebulizer, enough heating could be applied to the liquid drawn from the reservoir sufficient to create a vapor in the fluid flow path. In that case, nebulization of the now already vaporized material is not necessary, so that neither a highly pressurized air supply path (from a pump or compressed source) nor a high-velocity air stream is required. In that case, the air stream from the nozzle 62 is merely provided to mix with the vaporized material and help direct that mixture out of the chamber 53 to a user.

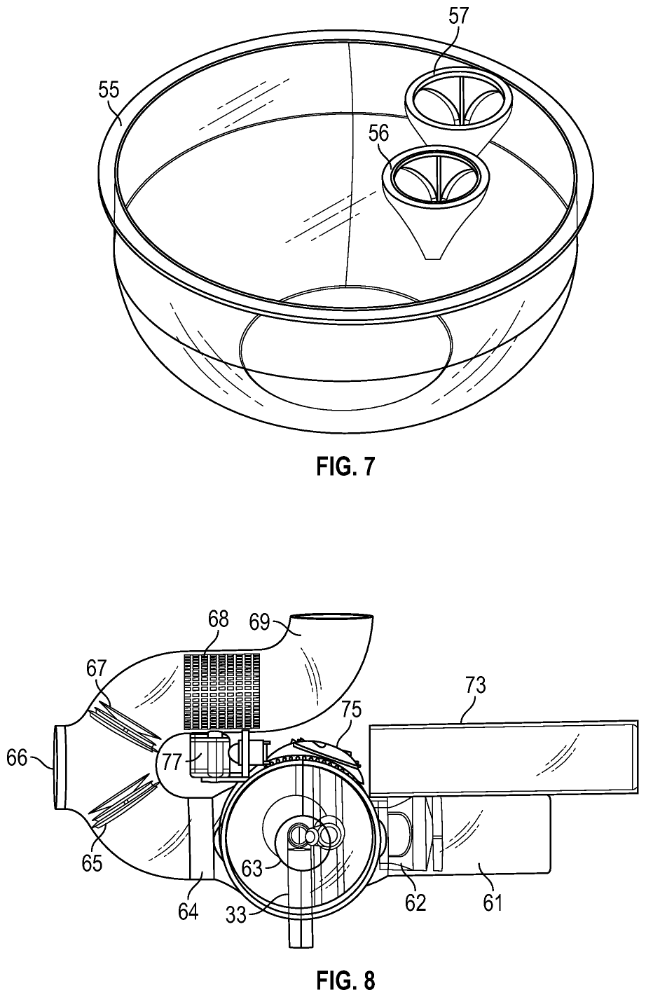

Also, the liquid reservoir can be provided in the form of an attachable reservoir cartridge 55, instead of simply storing the liquid at the bottom of the chamber 53. Not only does this prevent sloshing of liquid about the chamber 53 but, in the case of heated devices like that shown in FIG. 6, more effectively isolates the liquid material from unnecessary heating until it is drawn up through the flow path 54. The bottom 52 of the chamber 53 can be detached to allow insertion of a new cartridge 55 therein. As seen also in FIG. 7, the cartridge 55 may have a set of check valves 56 and 57 that can prevent leakage of liquid from the reservoir 55 in the event the cartridge were to be tilted or inverted, while still allowing adequate flow of liquid material into a fluid flow path 54 and admission of replacement air into the cartridge 55 to prevent vacuum lock.

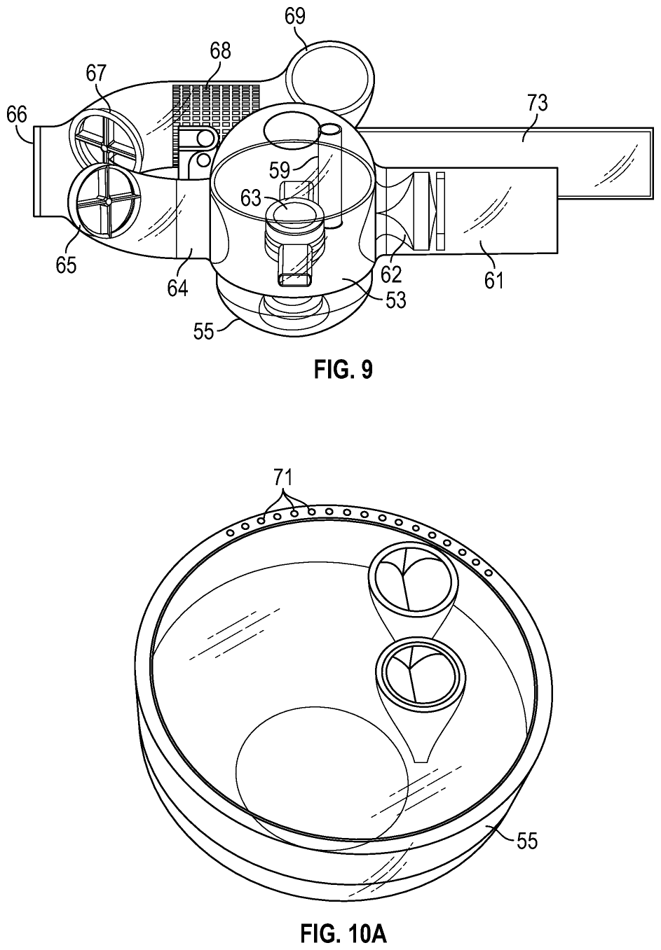

As in the previous embodiments, and as also seen in FIGS. 8 and 9, an inlet port 61 with check valve and Venturi nozzle 62 produces a high velocity airstream for carrying nebulized material drawn from the reservoir cartridge 55 out of fluid flow path opening 40 and nebulized by pressurized air source 33 in FIG. 8, or alternatively for carrying vaporized material drawn from the reservoir cartridge 55 out of the top vaporizer opening 63 of the heated fluid pathway 54 in FIG. 9.

The one-way check valve and Venturi nozzle 62 may together comprise a duckbill valve, which is an option for any of the embodiments and will be discussed further below. An air intake 59 admits air from the top, sides or bottom of the chamber 53 and into the cartridge 55 through the check valve 57. A discharge port 64 exits the chamber 53 and leads through a check valve 65 and an air pathway 66 to a user mask (not shown). Exhaled air is directed from the user mask through the air pathway 66 and check valve 67 to a filter 68 and outlet port 69.

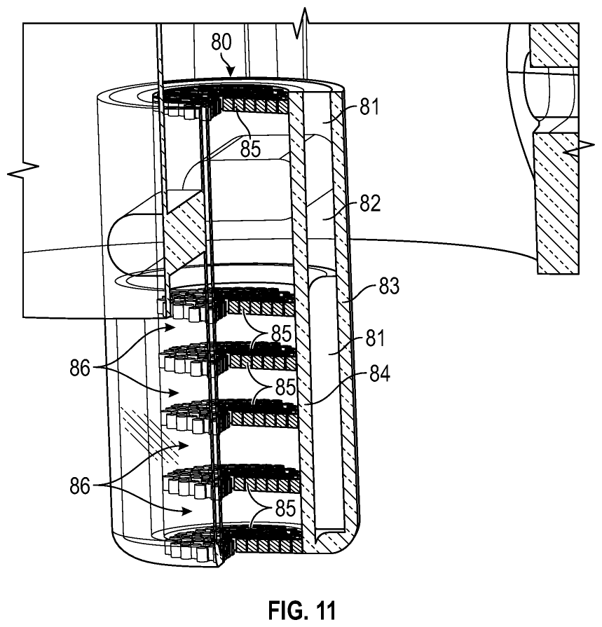

As seen in FIG. 6 (but also in more detail in FIGS. 11 to 14, discussed further below), multiple discrete heating elements 71 are spaced around the fluid flow path 54. In this way, the liquid drawn up through the flow path 54 may be heated prior to nebulization at the top opening 63. The liquid could be heated, e.g., close to normal human body temperature (37° C.) to ease the body's response to the aerosol being inhaled into the lungs. This reduces the chances of lung spasms in response to inhaling a cold aerosol mist, facilitates better bio-uptake of the intended medicinal material in the lungs (e.g. the body responds better to certain anesthetics if they are at body temperature), and more generally increases user comfort. Note that the Venturi effect itself causes the airstream to chill as it is accelerated by the nozzle 62 and then directed across the opening 63, so preheating of the liquid drawn through the flow path 54 is beneficial to restoring a more useful and comfortable temperature. Still further, heating of the liquid reduces the vapor pressure and thereby enhances nebulization efficiency. Finally, assuming the liquid material is adequately volatile, so that overly hot temperatures are not required, the heating can actually vaporize the material as it is drawn up through the flow path 54 for mixing with the airstream from the nozzle 62. It could then subsequently re-condense into an aerosol mixture as it interacts with the airstream and cools.

Since the heating elements require electricity and corresponding electrical and thermal control, inlet ports for the electrical pathways will be provided. A lithium ion battery pack 73 could supply the electrical power for the controlled heating, as seen in FIGS. 8 and 9, where for example the battery pack 73 is conveniently attached to the inlet port 61. A control circuit board 75 and a thumb activated trigger switch 77 (in some embodiments including a fingerprint sensor to prevent unauthorized use) could likewise be attached at any convenient location on the exterior of the device. In some embodiments the control circuit board 77 could require activation of the user's authorized fingerprint at a point-of-sale location or other location approved to verify the user's government issued ID. This would serve to prevent device usage by underaged or non-prescription users.

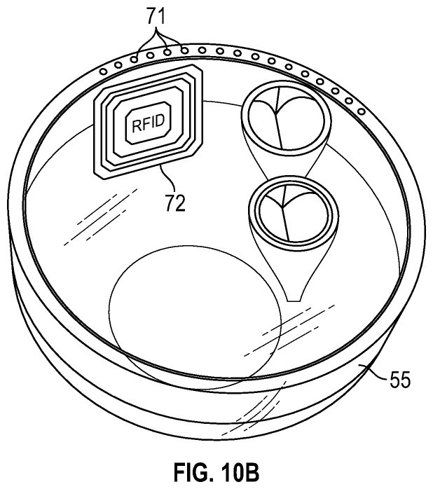

In one possible embodiment, a readable barcode or punch code 71 can be provided on the cartridge, for example on its edge as seen in FIG. 10, to provide a variety of information specific to the cartridge contents to the heater control 75. This can include heating parameters for the liquid material (such as specific heating zones or profiles of the discrete heating elements around the flow path, maximum temperatures, etc.).

In yet another embodiment, as seen in FIG. 10B, an RFID tag 72 could be included in the cartridge. The nebulizer's or vaporizer's heater control circuit could write to the RFID tag 72 via micro-USB, Bluetooth/WIFI connectivity, or other communication means to record cartridge information updates. Hence, the RFID tag 72 would not only allow storage of much the same kinds of coded information content as the barcode (e.g. specific parameters for heating the cartridge's liquid contents along the flow path) but could also log new information (such as the number of times the cartridge is used or whenever it becomes empty) to prevent unauthorized refilling of a cartridge. Stored information can include manufacturer authorization and batch codes, whereby a heater control circuit could activate a “limp mode” to prevent heating of unknown or adulterated contents. If the RFID coded information does not match manufacturer specifications (e.g. with a cartridge forgery), or the number of recorded uses exceeds some specified reasonable limit, or the cartridge has previously been empty but not refilled by the manufacturer itself, but by some unknown third party, then for user safety the nebulizer or vaporizer, responsive to the RFID coded information could refuse to operate.

It is anticipated that the additional information and functions described for RFID could alternatively be embodied utilizing flash memory written to by a direct physical interface. The interface might be via a male/female header pin bus pair, or by a contactor spring and contact, such as microUSB or microSD, with protocols defining data (such as SPI configuration), data clock, and power channels of the bus configuration.

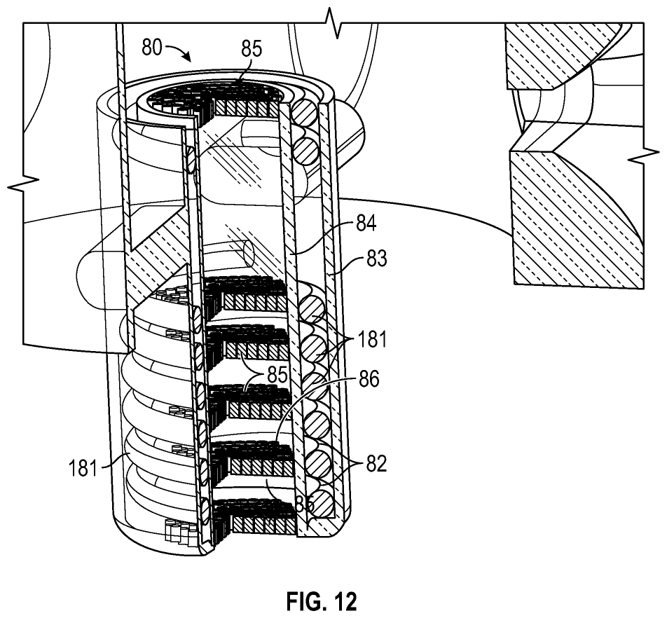

With reference to FIGS. 11-14, multiple discrete heating elements 81 (in this instance, two) surround the core of the fluid pathway 80 to provide gradated levels of heating. The heating elements 81 may be foil or solid rings or could be heating coils 181 as seen in FIGS. 12-14. Spacing 82 between the heating elements 81 reduces heat-soak between elements. An insulating outer liner 83 of ceramic, polyimide or other thermal insulation material may be provided to prevent heat transference into the fluid reservoir itself or into the duckbill valve or other Venturi nozzle where excess heating could cause damage. Only the fluid flow path 80 and the liquid within it should be heated by the elements 81. A liner 84 may be disposed between the heating elements 81 and the flow path 80. This inner liner 84 can serve as a thermal conductor (e.g. stainless steel) or as an insulator (e.g. ceramic or polyimide) depending on specific design intent (e.g., some portions of the liner along the length of the pathway 80 may be conductive and other portions may be insulative to precisely control where the heat is to be transferred into the liquid material, while keeping the liquid in the reservoir cool). Heating mesh 85 is in the fluid path 80 to conduct heat from the liner wall 84 into the center of the flow path 80. This added thermal conductivity removes any need to overheat the liquid along the wall 84 of the passage 80 to compensate for cooler liquid passage along the center of the passage 80. The mesh 85 could instead be in the form of a lattice, coils or filamentary material. It is anticipated that wicking material 86, commonly used in standard vaporizers, could be packed between the heat-conducting mesh/lattice/coils/filaments to assist moving the fluid up through the pathway 80. Since the inner liner 84, the mesh 85, and wicking material 86 are in contact with the liquid material to be nebulized and inhaled by a patient, they will need to be composed of bio-compatible materials to avoid any cross-contamination.

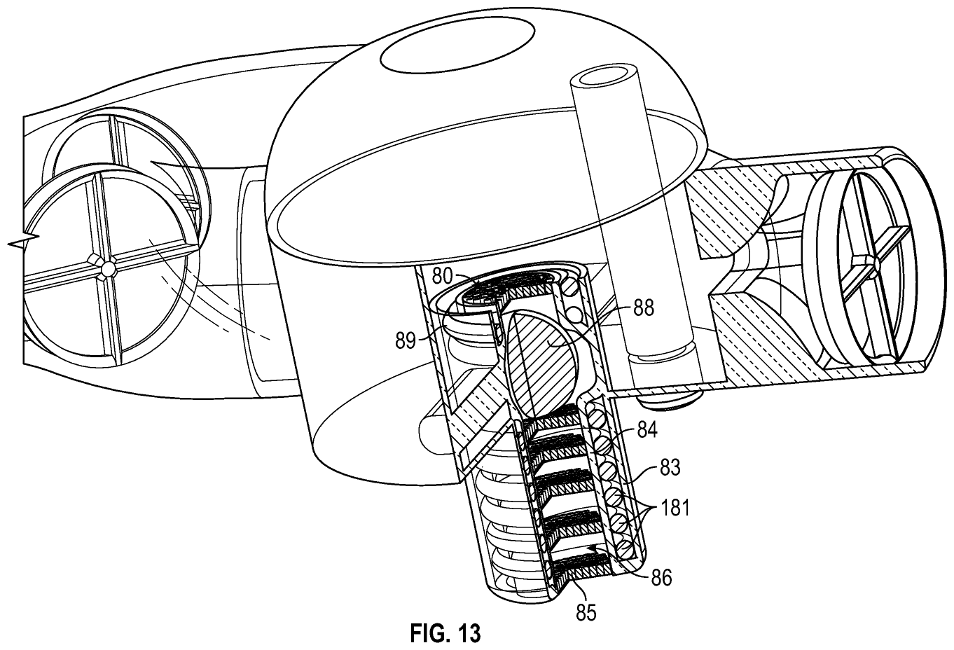

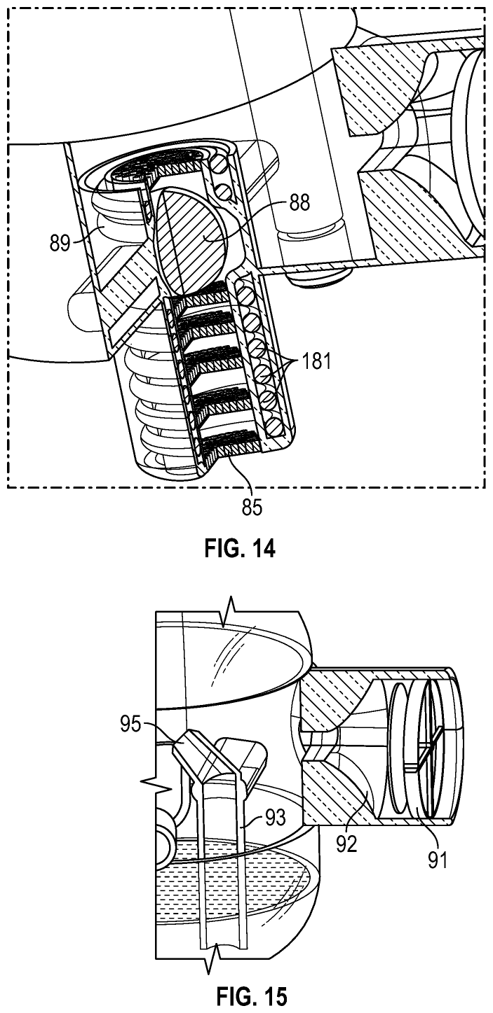

As seen in FIGS. 13 and 14, a ball valve 88 may be part of the fluid supply pathway 80. A weighted ball normally resting one of the conducting mesh elements 85 to allow fluid to pass around the ball, will engage a sealing surface 89 if the device is inverted to prevent leakage of liquid out of the reservoir and flow path. A similar ball valve may also be included in the air return tube. An added advantage to having the ball valve in a heated nebulizer (or vaporization) device is that the ball 88 will be pushed upwards against sealing surface 89 if vapor flow is very strong and therefore act as a check against too hot material from being inhaled and burning the mouth, throat or lungs of a patient.

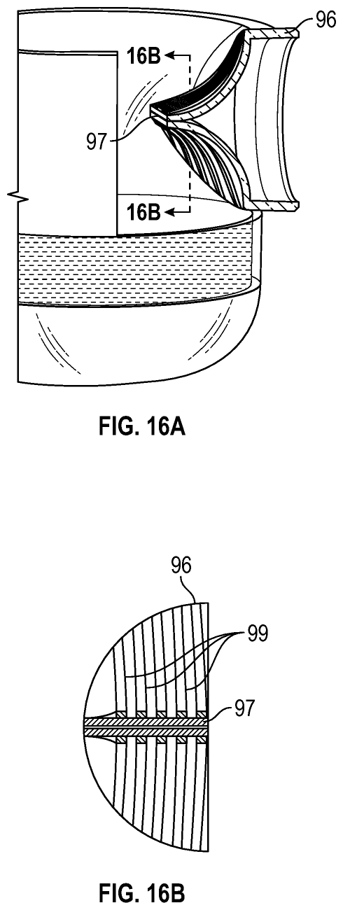

With reference to FIGS. 15 and 16, two versions of the check valve and Venturi nozzle are shown. In FIG. 15, a check valve 91 and Venturi nozzle 92 are separate components and the Venturi nozzle 92, while serving as a partial check valve, is mainly provided for its acceleration of the incoming airstream into a directed high velocity stream across the top opening 95 of the fluid flow path 93. Since some liquid could leak through the nozzle 92 if the device is tilted, the check valve 91 is provided to block any liquid from splashing out of the nebulizer device. Alternatively, in FIG. 16, the check valve and Venturi nozzle are combined into a single duckbill check valve 96 whose beak 97 has a sufficiently narrow opening that liquid cannot substantially leak out. The duckbill valve 96 is shaped to serve the dual function as a nozzle that accelerates airflow over the supply pathway's opening. It has a nozzle shape to create a Venturi effect on air flowing through it. To enhance its performance, a set of ribs 99 parallel to the airflow may be provided on the interior throat or bill of the valve 96 to ensure laminar flow toward the beak opening, as seen in the FIG. 16B cross-section. Additionally, the beak opening 97 of the valve 96 may, in some cases, be thickened to form a ring of material around the opening 97 that will maintain the widened shape of the opening to create the ribbon of accelerated laminar-flow air (rather than stretching into an annular shape) as well as make it more rigid and avoid any vibratory opening and shutting of the opening.

### Cartridge/Nebulizer Activation and Operation

The cartridge containing liquid contents to be nebulized (or vaporized) interacts with a control circuit of a nebulizer (or vaporizer) device to facilitate (or prevent) access to the cartridge contents through the device according to firmware instructions and stored information in nebulizer flash memory and/or cartridge RFID or edge barcode or pin-code information.

The nebulizer stored information could include a list of approved cartridge manufacturer codes, a list of “pirated” third-party establishment activation codes that are known to have been used contrary to agreed nebulizer policy or in violation of criminal/civil laws, an encoded biometric scan “avatar” that is unique to a specific nebulizer user created during third-party activation (to prevent its use by anyone else), usage limitations, and (for those nebulizers and vaporizers with heating elements) a generic heating set point and/or specific heater band parameters established during third-party activation (e.g. by a pharmacist).

Examples of biometric information that could allow a unique individual to be recognized include fingerprints, iris or retinal images, and facial images. It might also possible that unique electrocardiogram pulse patterns and electroencephalogram neural profiles could be employed and paired with a wearable watch or ring, or an EEG headset or brain-computer interface (e.g., hardware manufactured by Emotiv Inc.). A nebulizer/vaporizer device would be equipped with, or alternatively paired via wireless networking technology to, a biometric scanner. The nebulizer's controller would then confirm that the biometric data received from the scanner matches that previously stored at the time of activation before the cartridge and nebulizer could be used.

Cartridge edge barcode or pin code information can include a manufacturer code, a lot/batch number, an expiration date and/or expiration period after first usage (to prevent old cartridges with spoiled contents from being used), a maximum anticipated usage limit (beyond which replacement or an authorized refill of the cartridge is the only option), an age restriction (to be verified at time of third-party activation of the cartridge), a daily/hourly maximum usage frequency and/or dosage limit (to prevent taking medication too frequently and potential overdoses by patients who may forget “did I take my medicine today?” or to help prevent addiction), and heating parameters for the specific cartridge contents. These parameters can include a specific set point or maximum temperature for the material and heater ramp-up multiplier (how quickly can the temperature be brought up to set point by applied heating), and an optimal specific temperature for each heating band wherever multiple heating bands are employed by the nebulizer/vaporizer device.

Cartridge RFID information can include any of types of information as the edge barcode or pin code, but additionally a third-party establishment activation code (providing traceability to a specific doctor, pharmacy, medical marijuana dispensary, tobacco retailer, or authorized point-of-sale site) plus the aforementioned biometric “avatar” unique to the prescription holder or other intended user that is stored at the time of third-party activation of the cartridge (to be subsequently matched by the nebulizer device prior to each use). This helps to ensure that the intended user is the only person able to use that specific cartridge, even if the nebulizer or cartridge is stolen. Because no central database of patient personal information would be created at third-party activation, and the biometric “avatar” information is stored only in the cartridge, personal privacy is preserved. Additionally, a usage log in the form of a date/time stamp of initial use and each subsequent use of the cartridge can be recorded onto the RFID tag by the nebulizer device. Likewise, for cartridges employing a flash memory, the information could be the same as in RFID, except that the data would be written via a direct physical interface instead of the radio frequency signals of RFID. Flash memory might be used especially (but not necessarily exclusively) for those embodiments where a cartridge is permanently attached to (not removable from) the nebulizer and needs to be reactivated with each refilling with new liquid contents.

For cartridges containing an edge barcode, an optical scanner is provided in the nebulizer/vaporizer device to read the barcode information. This may be in the form of small LED emitter/detector pairs to read the printed stripes of the barcode. The optical reader could be located on an edge of the heater control circuit in direct proximity with the edge of the cartridge to allow interaction between the LED emitters, barcode stripes and the detectors that sense light reflection.

For cartridges containing an edge pin code, the coded information would be in the form of pinholes on the edge of the cartridge and the nebulizer/vaporizer device would be provided with electrical pin contactors (physical pin switches) also located on an edge of the heater control circuit in direct proximity to the cartridge edge to allow electrical contact with the pinhole locations. The presence of pinholes on the cartridge edge would create an open switch, while their absence would close the switch of the contactor elements on the device.

For compatibility, cartridge edges would have one standard location for any barcode and a different standard location for any pin code. Both edge barcodes and pin codes are very cheap and easy to implement, but their information carrying capacity is limited compared to RFID and not writable in use.

For cartridges containing an RFID tag, an RFID reader/writer board can be in a pocket in the bottom of the nebulizer chamber located directly above the position of the cartridge RFID tag. One example of an RFID reader/writer module is MFRC522 commercially available from RobotDyn of Zhuhai China. With that module, the reader and the tags communicate using a 13.56 MHz electromagnetic field via an antenna built into the board. Similar RFID reader/writer modules are available from other suppliers. Alternatively, because reading and/or writing can be robustly achieved even at distances greater than one inch (25 mm) away from the RFID tag, the RFID reader/writer might be embedded in the device heater control board and be protected within the nebulizer/vaporizer device.

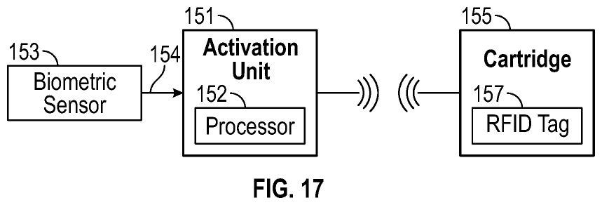

With reference to FIG. 17, a cartridge activation system is seen to have an activation unit 151 having a processor 152 running a sequence of programmed instructions. A biometric scanner 153 is coupled to the activation unit 151, for example via either direct wire connection 154 or Bluetooth or Wi-Fi network communications. A cartridge 155 with an initially unwritten RFID tag 157 interfaces with the activation unit 151 for receiving coded information that is to be written into the RFID tag 157. The programmed instructions executed by the processor 152 of the activation unit 151 may include using the biometric scanner 153 to create a unique biometric avatar of an intended cartridge user and writing said avatar to the RFID tag 157 together with an activation code associated with an authorized establishment. Alternatively, the data could be written via direct connection to a flash memory (not shown) on the cartridge 155 in the same manner as writing to the RFID tag 157.

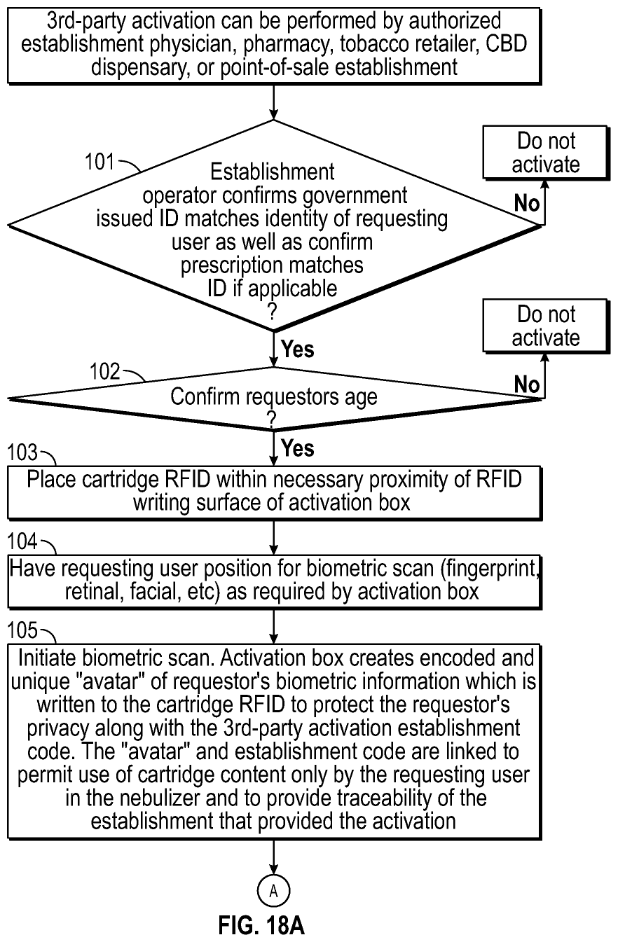

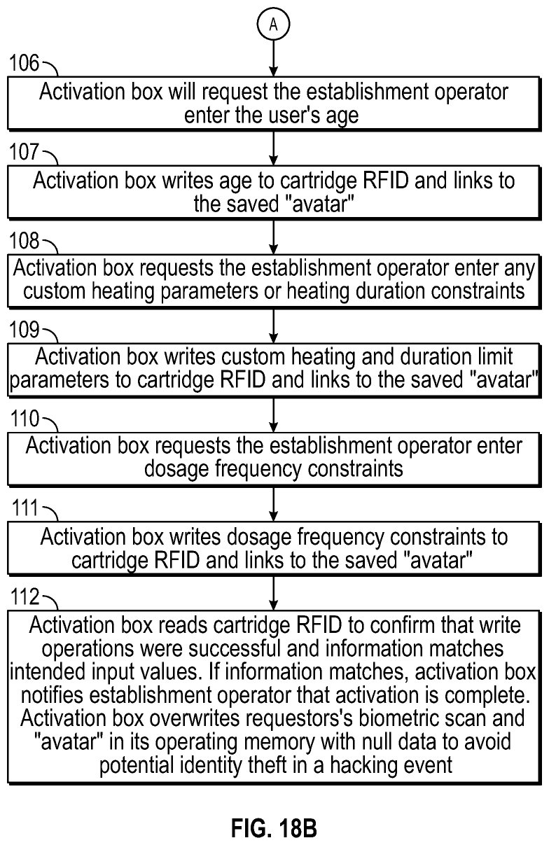

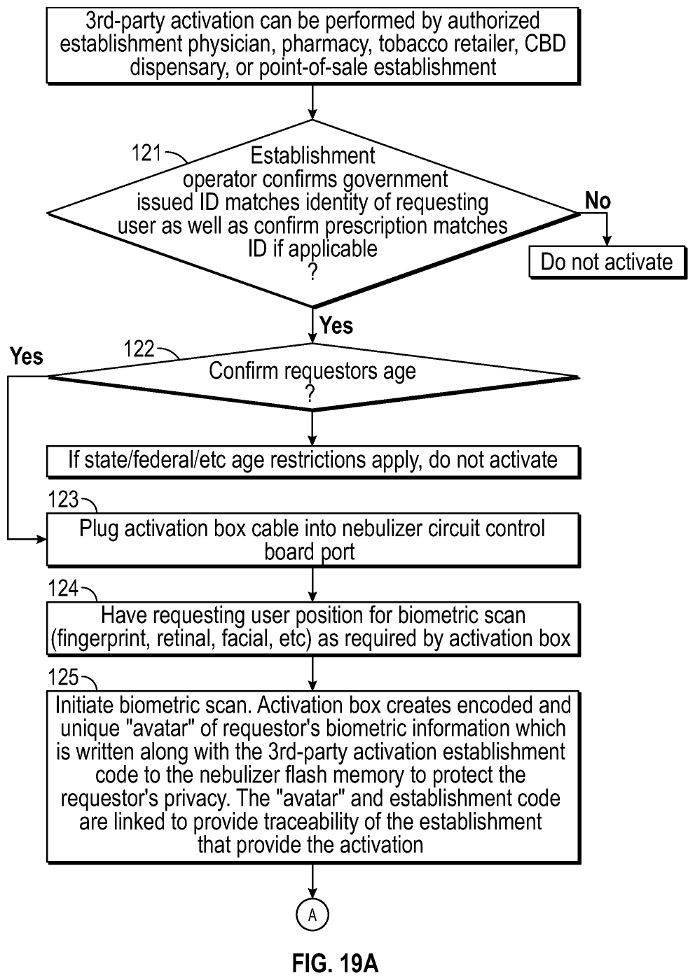

With reference to FIG. 18, a third-party activation flowchart shows that activation of a cartridge for a nebulizer/vaporizer device can be performed by an authorized establishment (e.g. a physician, a pharmacist, a tobacco retailer, medical cannabis dispensary, or other approved point-of-sale establishment). To activate, an establishment operator first confirms 101 that the requesting user has valid government-issued identification that matches his person and, where applicable, a prescription for the cartridge contents. Persons without ID may be refused activation of the product. Likewise, for prescription medicines, a person without a prescription matching his/her identity will be refused activation of the product. If the substance has age restrictions (e.g. for adult use only), the establishment operator would also verify 102 the intended user's age from the ID. Anyone not meeting the age requirements would be refused activation. Because activation includes recording of the identification code of the authorized establishment with the cartridge (in the RFID tag) and/or nebulizer/vaporizer device (in flash memory), any wrongful activation can be traced back to that establishment. Activation involves, for example, placing 103 the cartridge RFID tag within the necessary proximity (e.g. 25 mm) to an RFID writing surface of an activation unit at the establishment.

The activation will typically also involve the recording of a biometric “avatar” of the intended user so that only that individual can have access through the nebulizer to the cartridge contents. The establishment operator would therefore request 104 that the user position him/herself for biometric scanning, as required by the activation box. The activation box itself (with embedded scanner) or a biometric scanning device paired to the activation device (e.g. via a physical or wireless communication link) will then initiate the biometric scan 105 and thus create/encode a unique “avatar” of the requestor's biometric information and write it to the cartridge RFID tag, along with the third-party establishment's activation code. The establishment code and avatar are thereby linked within the cartridge's RFID tag to permit only that user to use the cartridge, and the issuing establishment is traceable. Where necessary, the activation box will also request 106 that the establishment operator enter the user's age, which can then likewise be written 107 to the RFID tag and linked the saved establishment code and user avatar.

In the event the cartridge does not already have stored heating parameters associated with the cartridge contents, such as when a pharmacist fills (or refills) the cartridge at the establishment instead of using a prefilled cartridge from the manufacturer, the activation box will request 108 that the establishment operator enter custom heating parameters or duration limits, which is then stored 109 in the RFID tag. This will ensure that the contents will not be overheated during use. Likewise, the activation box will request 110 that the establishment operator enter the required dosage restraints (including dosage frequency), which is then written 111 to the cartridge RFID tag. This ensures that a user only receives the proper dosage at the proper interval.

Once all activation has been written, the activation box will read 112 the cartridge RFID to confirm that the write operations were successful, and information matches the intended input values. If the information matches, the establishment operator is informed that activation is complete. To avoid potential identity theft from a hacking event, the requestor's biometric “avatar” data in the activation box's operating memory is immediately overwritten with null (or randomized) data. Alternatively, all avatar creation and handling during activation can be performed using RAM only in the activation box (e.g., preferably SRAM, but alternatively DRAM or other volatile memory despite their theoretically longer data retention times) so that de-powering of the activation unit (or at least of the avatar-handing SRAM circuitry therein) ensures all data is permanently lost.

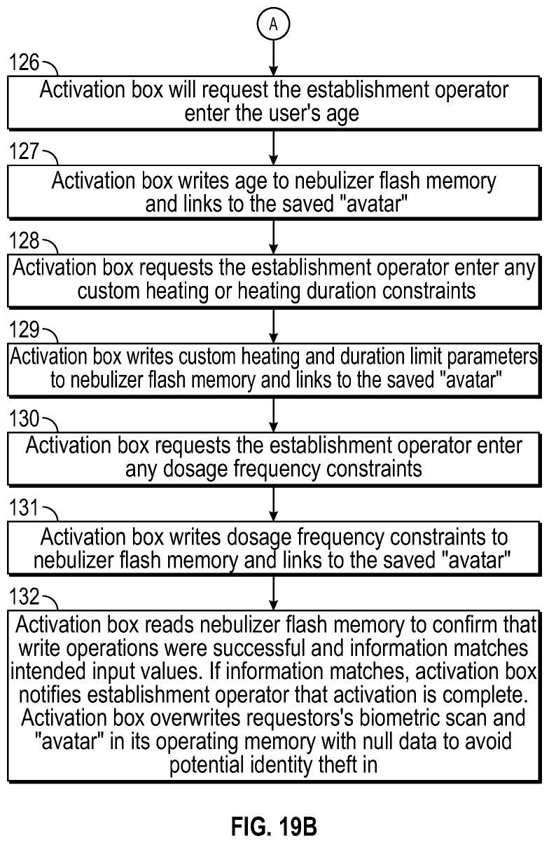

With reference to FIG. 19, a similar activation procedure is used for a nebulizer/vaporizer device, by writing to the nebulizer's flash memory. Whenever a user purchases a nebulizer, third-party activation of the device by an authorized establishment (e.g. physician, pharmacist, medical cannabis dispensary, tobacco retailer, or other authorized point-of-sale establishment) begins with verification 121 of the intended user's identity. If the nebulizer itself is a prescription product (or comes filled with a prescription medication), confirmation of the prescription will also be required. For age-restricted products, confirmation 122 of the user's age from government-issued ID will also be required prior to activation of the device. The nebulizer's control circuit board port will be plugged into or otherwise set to interface with the activation box 123. Having requested 124 a user to position him/herself for biometric scanning, a scan creates 125 a unique “avatar” of biometric information for the user, which is then recorded into the nebulizer flash memory. The establishment's authorization code is also recorded in the flash memory to permit traceability of the activation. Likewise, the establishment operator will enter 126 the user's age, which is written 127 to the nebulizer's memory and linked to the “avatar”. Heating constraints may likewise be requested 128 and written 129 to allow the nebulizer/vaporizer device to appropriately control heating of its contents. Similarly, dosage constraints (including frequency of use) may be requested 130 and written 131 to the flash memory. After all required information has been written to the nebulizer flash memory, the activation box will typically read 132 the recorded information to verify that writing was successful and matches the intended input values. Activation of the device is now complete. Again, as with the activation of cartridges, the requestor's biometric “avatar” data in the activation box's operating memory will be overwritten to ensure user privacy and security.

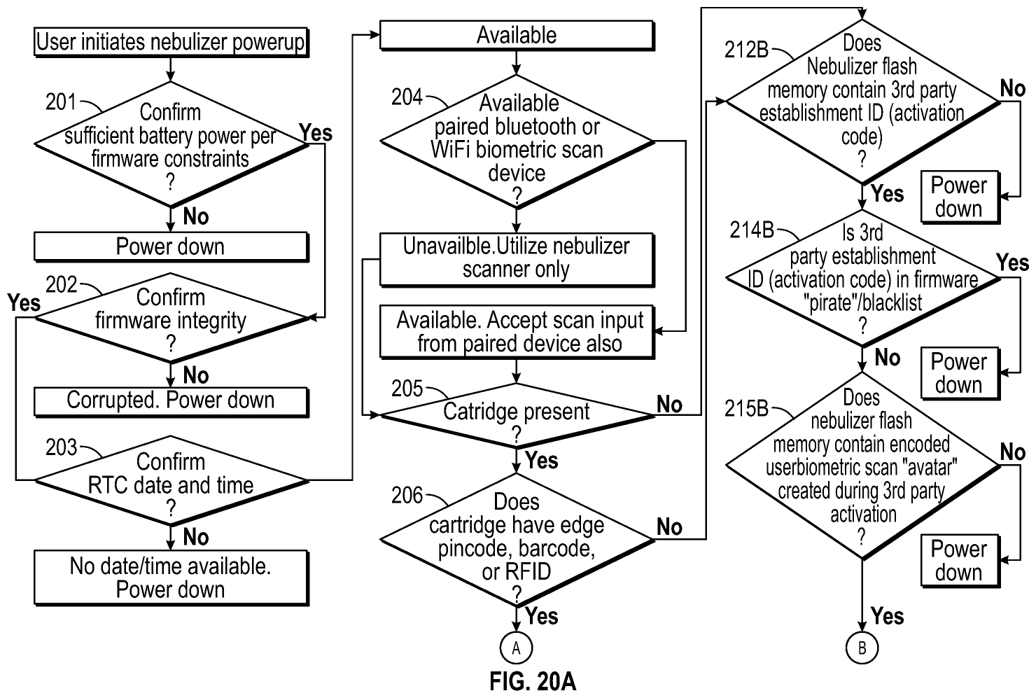

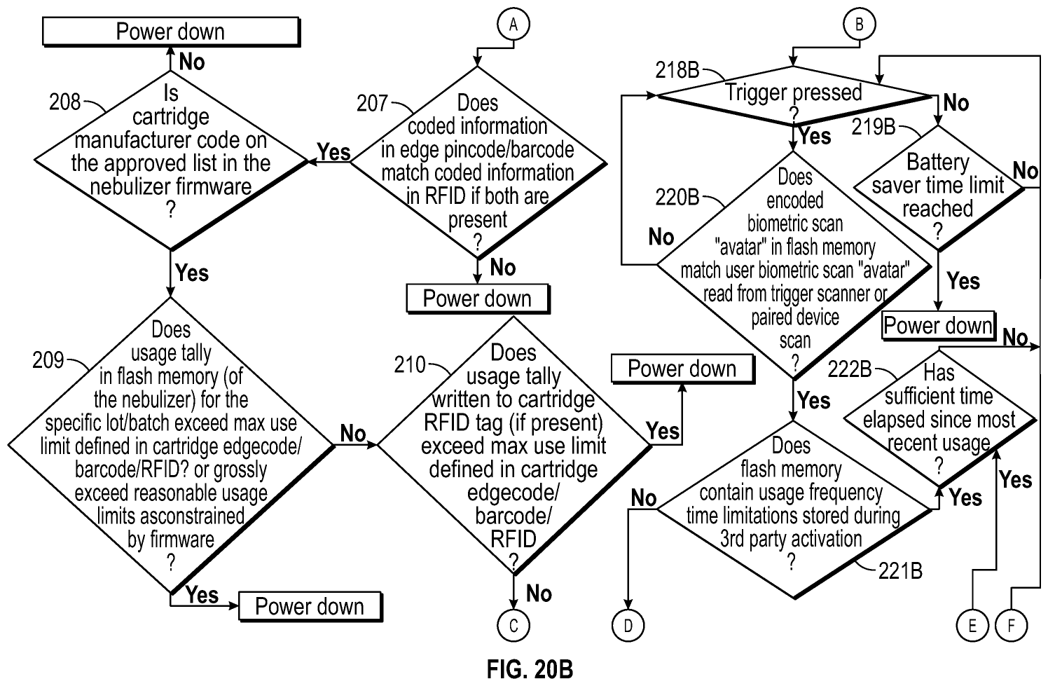

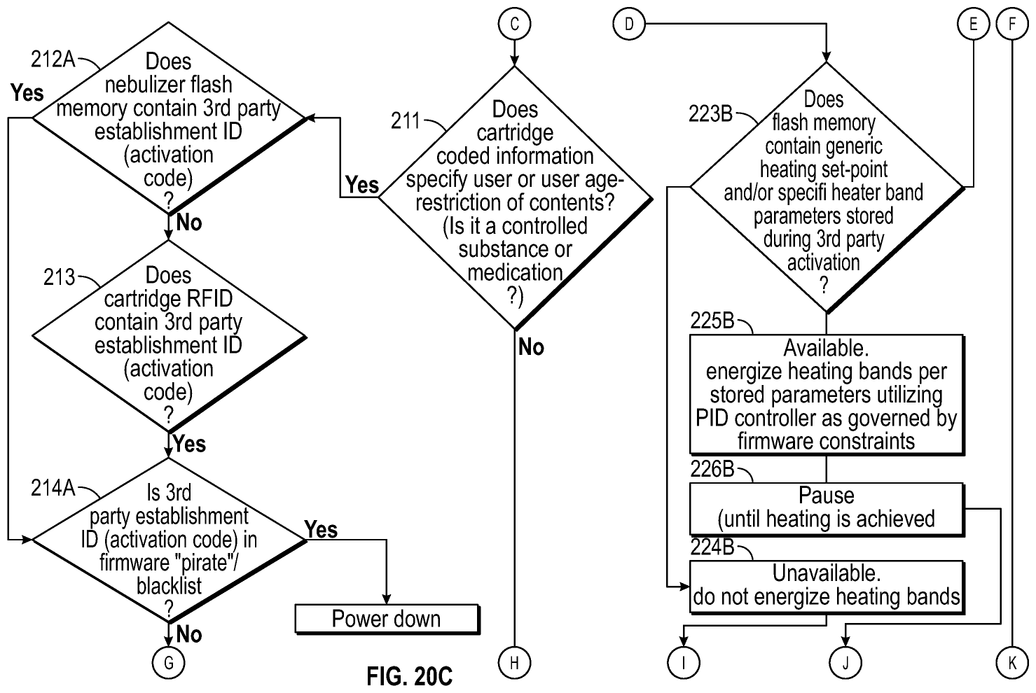

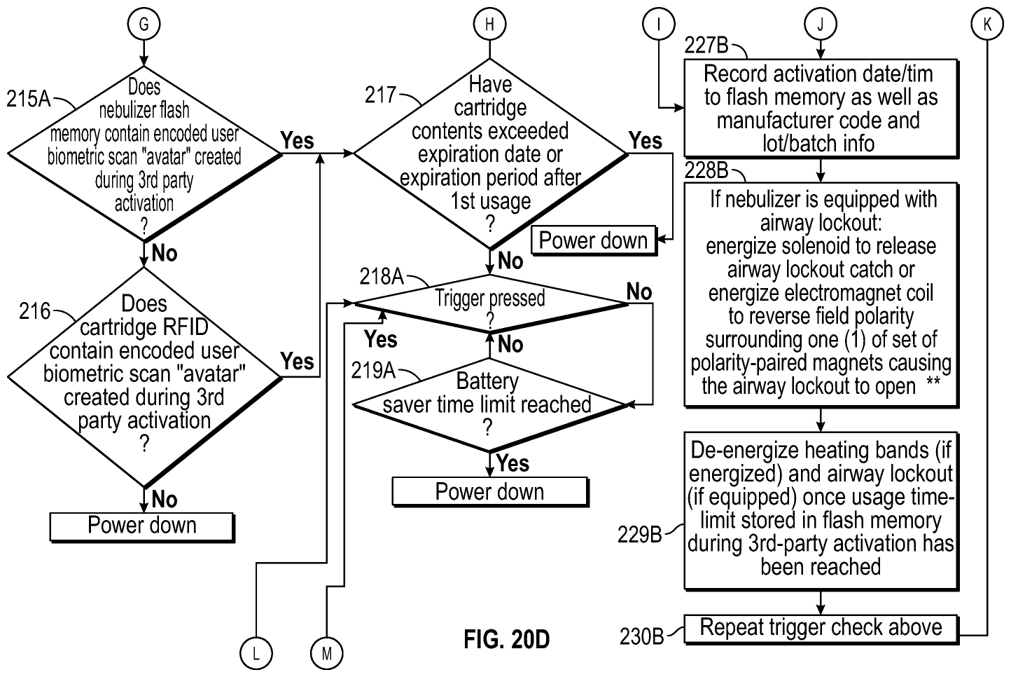

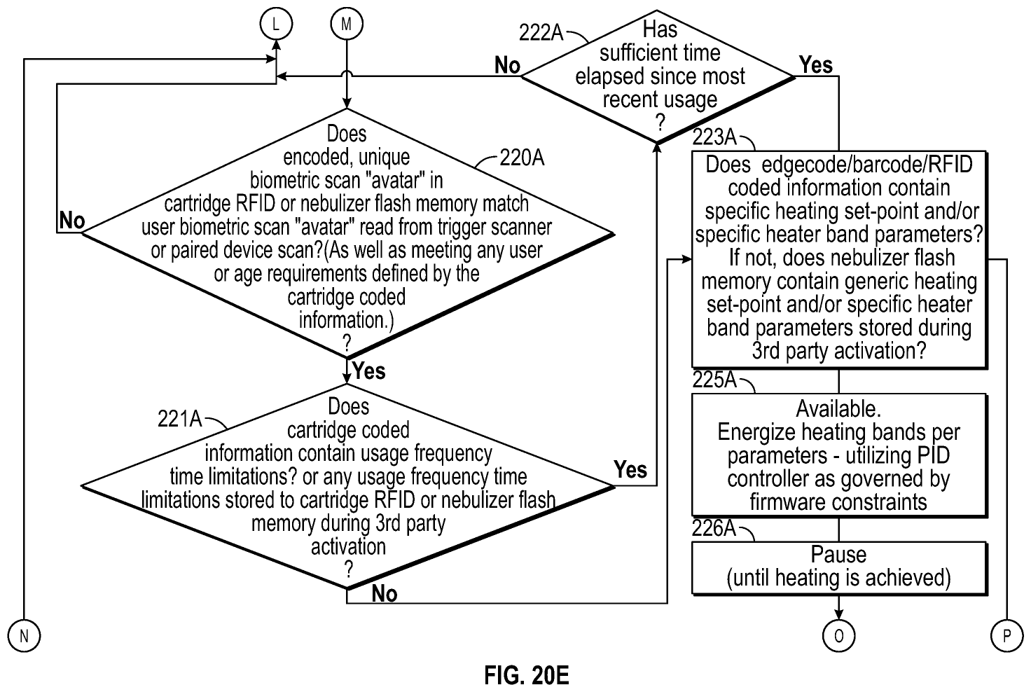

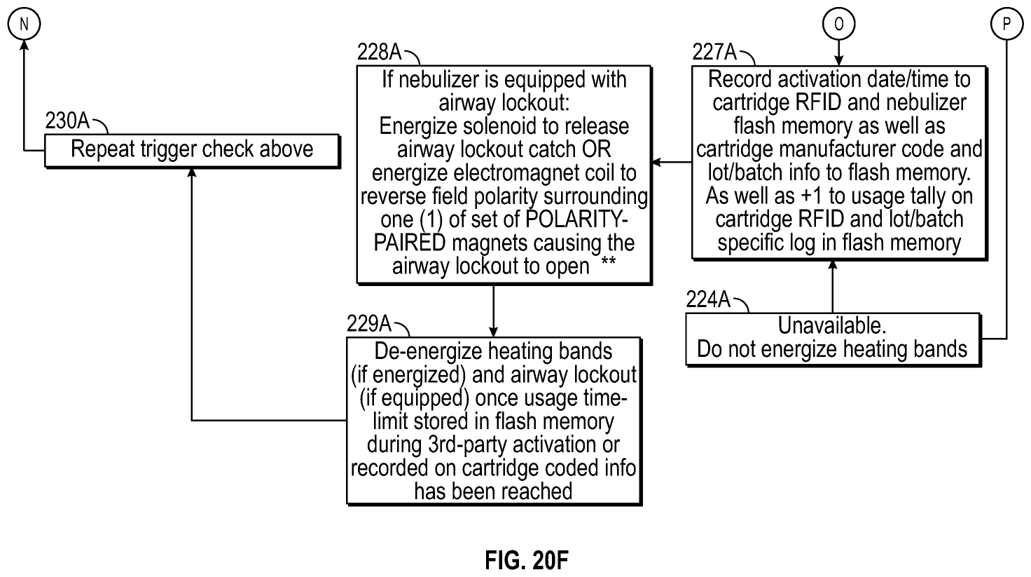

With reference to FIG. 20, an operational flowchart illustrates use of an activated cartridge in a nebulizer/vaporizer device according to representative firmware instructions used by the nebulizer device's control board. The sequence begins with typical power-up procedures. Where the nebulizer device is battery operated, the firmware confirms 201 that there is sufficient battery power available per preset constraints. Firmware integrity is confirmed 202. The real-time clock (RTC) date and time is confirmed 203 as being available and correct. The presence or absence of biometric scan device paired to the device is confirmed 204. If paired biometric scanner is available it will be used to verify user identity; otherwise, a scanner embedded in the nebulizer will be used. If any of these checks fail, the unit will power down. (Corresponding error codes may be recorded for diagnostic purposes.)

The presence of an installed and activated cartridge is confirmed 205. The firmware may be general enough to be used both with cartridge-only nebulizers and with nebulizers having their own fillable content volume. Likewise, the firmware could accommodate nebulizer devices that allow use of cartridges lacking their own edge barcode or edge code and/or RFID tag (checked in step 206).

When a cartridge is used that has encoded information in either edge barcode or pin code, and/or an RFID tag, a series of checks of that information follows. Does the coded information in the edge barcode or pin code match that in the RFID if both are present? (step 207) Is the cartridge manufacturer code on an approved list in the nebulizer firmware? (step 208) Does the usage tally in the nebulizer flash memory for the specific cartridge lot/batch number either exceed a maximum use limit specified by the cartridge information or grossly exceed (e.g. by some set percentage) “reasonable” usage limits constrained by the firmware? (step 209) Does the usage tally written to the cartridge RFID tag (if present) exceed the maximum use limit defined by the cartridge info (barcode, pin code, or RFID)? (step 210) If any of these queries is true, the unit will power down, preventing usage of unapproved or expired cartridges.

Next, a series of checks of the activation information follows before the unit can be used. Does the nebulizer flash memory contain a third-party establishment identification (activation code) (step 212A or 212B)? Does the cartridge RFID (if any) contain the third-party establishment activation code? (step 213) Is the third-party establishment activation code found in a firmware “pirate” blacklist? (step 214A or 214B) This would be the case if an establishment had violated rules of usage by, for example, selling products to underage users or without a prescription, or have been found to have refilled cartridges with contraband contents (or sold such cartridges). Does the nebulizer flash memory contain an encoded user biometric scan “avatar” created during third-party activation? (step 215A or 215B) If not, does the cartridge RFID contain such an “avatar”? (step 216) Have the cartridge contents passed the expiration date, or the expiration period after first usage? (step 217) If any of these checks fail, the unit will power down, preventing usage of not activated or improperly activated or expired cartridges or nebulizers.

The system checks to see whether the nebulizer's trigger has been pressed for the user to obtain nebulized or vaporized contents (step 218A or 218B) When the trigger is not pressed, the firmware regularly checks the battery saver time (on battery operated units) to see whether a time limit has been reached, and powers down if the device is not being used. (step 219A or 219B) When the trigger is pressed, further checks are initiated. Does the encoded biometric “avatar” in the nebulizer flash memory or cartridge RFID match that read from a paired device scan (or a from scanner embedded in the nebulizer control unit)? (step 220A or 220B) This verifies the identity of the user. The stored user age information in the flash memory or RFID tag is also checked, where necessary. Does the nebulizer flash memory or cartridge RFID contain usage frequency time limitations (step 221A or 221B), and if so has sufficient time elapsed since the most recent usage (step 222A or 222B)?

Does the cartridge coded information (barcode, pin code or RFID) or the nebulizer flash memory contain specific heater band parameters stored during manufacture or third-party activation, or if not does the nebulizer flash memory contain a generic heating set point? (step 223A or 223B) If such heating parameters are unavailable, heating elements will not energized (step 224A or 224B). Otherwise if available, these parameters will be used to energize nebulizer heating bands (if any) and control the heating per the accessed parameters (step 225A or 225B), utilizing a PID controller and governed also by firmware constraints until the required heating is achieved (step 226A or 226B).

Activation date and time are recorded to nebulizer flash memory and/or cartridge RFID and the usage tally is likewise incremented by +1 (step 227A or 227B). This recorded activation information normally includes the specific cartridge manufacturer code and lot/batch information.

Many nebulizer/vaporizer devices also include an airway lockout (e.g. a solenoid valve or polarity-paired magnetic coils) that needs to be activated in order to open the fluid pathway. After a set duration (e.g., defined by a maximum dosage for the specific contents), the heating bands will be de-energized and the airway lockout will be closed (step 229A or 229B) This maximum duration may be established during activation of the nebulizer device and/or cartridge. The firmware loops back (230A or 230B) to trigger depression and battery saver checks, and rechecking of the biometric avatar (steps 220A or 220B) so another user doesn't try to use the device, and a check of the time elapsed since last usage (steps 221-222A or 221-222B) so that an overdose can be prevented.

1. A cartridge for use with a nebulizer, comprising:
a reservoir containing a liquid having an outlet port with a first one-way valve connectable to a flow path of the nebulizer device to receive the liquid, the reservoir associated with a mechanical interface that joins the reservoir to the nebulizer;
an air intake with a second one-way valve to admit air into the reservoir while preventing leakage of liquid from the reservoir; and
a readable element storing coded information, the coded information being readable by a control circuit of the nebulizer.

2. A cartridge as in claim 1 wherein the readable element storing the coded information comprises a barcode or punch code provided on an edge of the cartridge.

3. A cartridge as in claim 1 wherein the readable element storing the coded information comprises a writable RFID tag.

4. A cartridge as in claim 1, wherein the readable element storing coded information comprises a flash memory with a direct connection interface.

5. A cartridge as in claim 1, wherein the coded information stored on the readable element includes at least information specific to liquid contained in the reservoir.

6. A cartridge as in claim 1, wherein the coded information stored on the readable element includes any one or more of a manufacturer-specific authorization code, a cartridge batch identification code, and a usage history log for the cartridge.

7. A cartridge as in claim 1, wherein the coded information stored on the readable element indicates any one or more of an unauthorized cartridge, a refilled cartridge, any nonapproved contents in the cartridge, and an unauthorized user that allows a control circuit of the nebulizer device to disable drawing of liquid contents from the reservoir.

8. A cartridge as in claim 1, wherein the readable element is also writable so as to receive and record coded information including any one or more of a third-party establishment activation code for the nebulizer and a biometric user identifier.

9. A cartridge as in claim 1, wherein the readable element stores a unique biometric avatar of an intended user that is linked to an authorized third-party establishment activation code.

10. A processor-controlled cartridge activation system for activating cartridges to be inserted into a nebulizer, comprising:
an activation unit having a processor running a sequence of programmed instructions;
a biometric scanner coupled to the activation unit; and
a cartridge with an initially unwritten memory that interfaces with the activation unit for recording coded information in the memory;
wherein the programmed instructions executed by the processor of the activation unit includes using the biometric scanner to create a unique biometric avatar of an intended cartridge user and writing said avatar to the memory together with an activation code associated with an authorized establishment, whereby a nebulizer reading the coded information from the memory can allow access to contents of the cartridge.

11. A cartridge as in claim 10 wherein the memory is in the form of an RFID tag interfacing with the activation unit via radio waves.

12. A cartridge as in claim 10 wherein the memory is the form of a flash memory with a direct connection interface to the activation unit.

13. A cartridge activation system as in claim 10, wherein the writing of the activation code and biometric avatar to the RFID tag provides a traceable certification that the intended user is authorized to access cartridge contents through a nebulizer to which the cartridge can be attached, the stored activation code and biometric avatar being readable from the RFID tag by such nebulizer.

14. A cartridge activation system as in claim 13, wherein the biometric avatar coded in the RFID tag is subsequently readable by a nebulizer to which the cartridge can be attached to allow access to cartridge contents through the nebulizer only to a unique user having a biometric avatar independently obtained by the nebulizer that matches the avatar that was stored in the cartridge RFID tag by the activation unit.

15. A cartridge activation system as in claim 10, wherein the programmed instructions further direct writing by the activation unit to the RFID tag of coded information specifying a content-specific dosage and usage frequency, the coded information being readable by a nebulizer to which the cartridge can be attached to allow user access to cartridge contents through the nebulizer only up to the dosage and usage frequency limits specified in the RFID tag.

16. A cartridge activation system as in claim 10, wherein the programmed instructions further direct writing by the activation unit to the RFID tag of coded information specifying content-specific heating parameters, the coded information in the RFID tag being readable by a nebulizer to which the cartridge can be attached to provide controlled heating of cartridge contents accessed by a user through the nebulizer in accord with the heating parameters stored in the cartridge RFID tag.
